(19) United States
(12) Patent Application Publication
(10) Pub. No.: US 2009/0198220 A1
(43) Pub. Date: Aug. 6, 2009

(54) OSMOTIC PUMP WITH AT LEAST ONE CHARGEABLE MATERIAL

(76) Inventors: Michel Paul Barbara Van Bruggen, Helmond (NL); Hendrika Cecilia Krijnsen, Boxtel (NL); Geert Langereis, Eindhoven (NL)

Correspondence Address: PHILIPS INTELLECTUAL PROPERTY & STANDARDS, P.O. BOX 3001, BRIARCLIFF MANOR, NY, 10510, US

(21) Appl. No.: 12/298,800

(22) PCT Filed: Apr. 18, 2007
(86) PCT No.: PCT/IB07/51391

§ 371 Date: Oct. 28, 2008

(30) Foreign Application Priority Data
Apr. 28, 2006 (EP) 06113248.6

(51) Int. Cl. A61M 5/145

(52) U.S. Cl. 604/892.1

(57) ABSTRACT

The invention relates to an osmotic pump (1) comprising at least one chargeable material (30) whereby the osmotic pressure is caused by inducing and/or de-inducing charges within the material.

The present invention is directed to the field of osmotic pumps.

Osmotic pumps for the controlled delivery of drugs e.g. inside a patient are widely known in the field. E.g. the WO 2005/032524 which is hereby incorporated by reference discloses an osmotic pump capable of delivering a drug with an ascending release profile.

However, in known osmotic pumps in the field there is the disadvantage that usually the osmotic pressure used to control the release of the drugs cannot be controlled in a sufficient and easy way. E.g. in the osmotic pump as disclosed in the WO 2005/032524 there is only the possibility that the delivery rate is increased over time whereas a decrease is impossible. Furthermore this control is pre-programmed and cannot be changed on demand, or remotely.

### SUMMARY OF THE INVENTION

It is an object of the present invention to provide an osmotic pump whereby the osmotic pressure can be easier controlled.

This object is solved by an osmotic pump according to claim 1 of the present invention. Accordingly, an osmotic pump for drug delivery, especially for drug delivery to and/or inside a patient is provided comprising at least one chargeable material whereby the osmotic pressure is caused by inducing and/or de-inducing charges within the material.

The term “chargeable material” means, includes and/or describes a material which is capable of being charged either negatively and/or positively, preferably by means of an electrochemical process.

By doing so, for most applications at least one of the following advantages can be achieved:

The osmotic pressure within the osmotic pump can be easily controlled simply by charging or un-charging the chargeable material; thus increasing or decreasing the osmotic pressure at will

The required energy is only small and is directly proportional to the amount of charged species formed

The required electrical potential will be only in the order of a few Volts

Especially because of the advantages listed above, the osmotic pump may in a wide range of applications be of use for drug delivery to and/or inside a patient.

According to an embodiment of the present invention at least one of the chargeable material(s) comprises a solid material.

According to an embodiment of the present invention at least one of the chargeable material(s) comprises a polymeric material.

According to an embodiment of the present invention at least one of the chargeable material(s) comprises a polymeric material with a density of ≧0.5 g/m³ and ≦2 g/m³

According to an embodiment of the present invention at least one of the chargeable material(s) comprises a polymeric material with a density of ≧0.75 g/m³ and ≦1.5 g/m³

According to an embodiment of the present invention at least one of the chargeable material(s) comprises a polymeric material with a density of ≧1 g/m³ and ≦1.25 g/m³

According to an embodiment of the present invention at least one of the chargeable material(s) comprises a solid material with a chargeability of ≧0 and ≦3000 Coulomb per cm³ of solid material.

According to an embodiment of the present invention at least one of the chargeable material(s) comprises a solid material with a chargeability of ≧500 and ≦2500 Coulomb per cm³ of solid material.

According to an embodiment of the present invention at least one of the chargeable material(s) comprises a solid material with a chargeability of ≧1000 and ≦1750 Coulomb per cm³ of solid material.

According to an embodiment of the present invention at least one of the chargeable material(s) comprises an at least in its charged form water-soluble material.

According to an embodiment of the present invention the at least one chargeable material is located in a first reservoir comprising a first semipermeable material for the inflow of water into said reservoir.

According to an embodiment of the present invention the permeability of the first semipermeable material is ≧10⁻²⁶ m² and ≦10⁻¹⁴ m²

According to an embodiment of the present invention the permeability of the first semipermeable material is ≧10⁻²⁴ m² and ≦10⁻¹⁶ m²

According to an embodiment of the present invention the permeability of the first semipermeable material is ≧10⁻²³ m² and ≦10⁻¹⁷ m²

According to an embodiment of the present invention the osmotic pump comprises a second reservoir in which the drug to be released by the osmotic pump is located and whereby a movable piston (or e.g. a deformable membrane) is provided between the first reservoir and the second reservoir.

According to an embodiment of the present invention the drug is selected from the group comprising sufentanil, fentanil, morphine, leuprolide acetate, insulin, psychotropics, contraceptive agents, growth hormones or other proteins, peptides, enzymes, genes, factors, hormones or mixtures thereof.

According to an embodiment of the present invention the second reservoir comprises a second semipermeable material through which the drug is released by the osmotic pump.

According to an embodiment of the present invention the permeability of the second semipermeable material is ≧10⁻¹⁶ m² and ≦10⁻¹⁰ m²

According to an embodiment of the present invention the permeability of the second semipermeable material is ≧10⁻¹⁵ m² and ≦10⁻¹¹ m²

According to an embodiment of the present invention the permeability of the second semipermeable material is ≧10⁻¹⁴ m² and ≦10⁻¹² m²

According to an embodiment of the present invention the at least one of the chargeable material(s) is a solid material selected out of the group comprising polypyrrole, polyaniline, polyacrylonitrile, polythiophene and mixtures thereof.

According to an embodiment of the present invention the second reservoir comprises a flow restrictor through which the drug is released by the osmotic pump.

A flow restrictor in the sense of the present invention means and/or includes a thin channel-shaped outlet from the second reservoir towards the outside.

According to an embodiment of the present invention, the length L of the flow restrictor is ≧0.01 cm and ≦10 cm, preferably ≧0.1 cm and ≦5 cm. It should be noted that the design of the flow restrictor may be straight, however, the flow restrictor may have any form such as curved or spiral-wound.

According to an embodiment of the present invention, the diameter of the flow restrictor is ≧10 μm and ≦500 μm, preferably ≧50 μm and ≦250 μm.

According to an embodiment of the present invention, the ratio of length L and diameter d of the flow restrictor is ≧200:1 and ≦50,000:1, preferably ≧2,000:1 and ≦10,000:1

According to an embodiment of the present invention the at least one of the chargeable material(s) is an at least in its charged form water-soluble material selected out of the group comprising alkylsulfonates, iodine, sulfides metals, preferably selected out of the group comprising Fe, Ni, Cu, Zn, Co, Al, Cr, Mo, Ru, Mn, Ir, Ag and mixtures thereof and their oxides, hydroxides, iodides, chlorides, sulfides, acetates, oxalates, phtalocyanines and mixtures thereof.

According to an embodiment of the present invention, the size of the charged form of chargeable material which is at least in its charged form water-soluble material is ≧0.5 nm and ≦10 nm.

According to an embodiment of the present invention, the first reservoir furthermore comprises an auxiliary salt component, which is preferably non-reactive during the performance of the osmotic pump.

According to an embodiment of the present invention, the auxiliary salt component is selected from the group comprising alkali and earth alkali chlorides, fluorides, bromides, iodides, sulfates, perchlorates, nitrates and mixtures thereof.

In some applications within the present invention, when there is a first solid chargeable material and a second chargeable material with a size as described above, it has been shown to be advantageous to add an auxiliary salt, which is believed (without being fixed to that explanation) to act as follows.

It is assumed that in the following the first solid chargeable material (=A) is oxidized and the second chargeable material (=B) is reduced during actuation of the pump, however, any skilled person in the art will easily see that the circumstances may very easily be reversed without any problem.

When actuating the osmotic pump, the first solid chargeable material (=A) and the second chargeable material (=B) will in most applications react as follows:

A =>A⁺+e⁻ (I)

B+e⁻=>B⁻ (II)

It should be noted that the equations (I) and (II) are simplified, especially in case that B is e.g. a disulfide, the reaction would be different; but in this regard, the reactions (I) and (II) are for explanatory purposes only and need not necessary resemble the actual events in the osmotic pump.

Due to the great size of the anion B⁻ it is hindered to wander directly to the cationic species A⁺, which is insofar preferred as it prevents the back-reaction of (II) to occur, which may happen in case that the anions B⁻ reach the solid material A.

In this case it is preferred that an auxiliary salt C⁺D⁻ is furthermore present in the first reservoir, so that salt couples A⁺D⁻ and C⁺B⁻ may form. In the overall reaction, which may be written very schematically like this:

A+B+C⁺+D⁻=>A⁺D⁻+C⁺B⁻ (III)

Two charged species are formed, thus increasing the osmotic pressure in the pump. Since the auxiliary salt is preferably non-oxidizable (and non-reducable) during performance of the osmotic pump, it is no problem that the salt couples A⁺D⁻ and C⁺B⁻ are formed since they will not react further.

The invention furthermore relates to a method of releasing a drug from a osmotic pump as described above whereby osmotic pressure is induced by charging the at least one chargeable material by use of electrical current.

The invention furthermore relates to the use of an osmotic pump as described within the present invention for drug delivery to and/or inside a patient.

The term “inside a patient” within the present invention means and/or includes especially that the drug delivery means may be implanted inside a patient.

According to an embodiment of the present invention, the osmotic pump is adapted to deliver drugs inside a patient intramuscular and/or intratumoural, and/or subcutaneous, and/or intravenous, and/or intravascular, and/or peritoneal, and/or intraspinal.

The term “to a patient” within the present invention means and/or includes especially that the drug delivery means may be outside a patient but is connected to the patient.

According to an embodiment of the present invention, the osmotic pump is adapted to deliver drugs to a patient subcutaneous and/or intramuscular and/or intratumoural and/or subcutaneous and/or intravenous and/or intravascular and/or peritoneal and/or intraspinal.

An embodiment of the present invention furthermore relates to the use of an osmotic pump as described within the present invention for long-time drug delivery to and/or inside a patient.

The term “long-time” within the present invention means and/or includes especially that a drug (or a solution containing a drug) may be applied to and/or inside a patient for a time which expands for several days, according to an embodiment several weeks, according to an embodiment several months, according to an embodiment one year and even longer time.

An embodiment of the present invention furthermore relates to the use of an osmotic pump as described within the present invention for drug delivery to and/or inside a patient, whereby the drug is selected from the group comprising sufentanil, fentanil, morphine, leuprolide acetate, insulin, psychotropics, contraceptive agents, growth hormones or other proteins, peptides, enzymes, genes, factors, hormones or mixtures thereof.

An osmotic pump according to the present invention may be of use in a broad variety of systems and/or applications, amongst them one or more of the following:
drug delivery systems
liquid absorbers
sample handling devices
micro valves
analytical devices
micropumps

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

Additional details, characteristics and advantages of the object of the invention are disclosed in the subclaims, the figures and the following description of the respective figures and examples, which—in an exemplary fashion—show one embodiment

of an osmotic pump according to a first embodiment of the present invention.

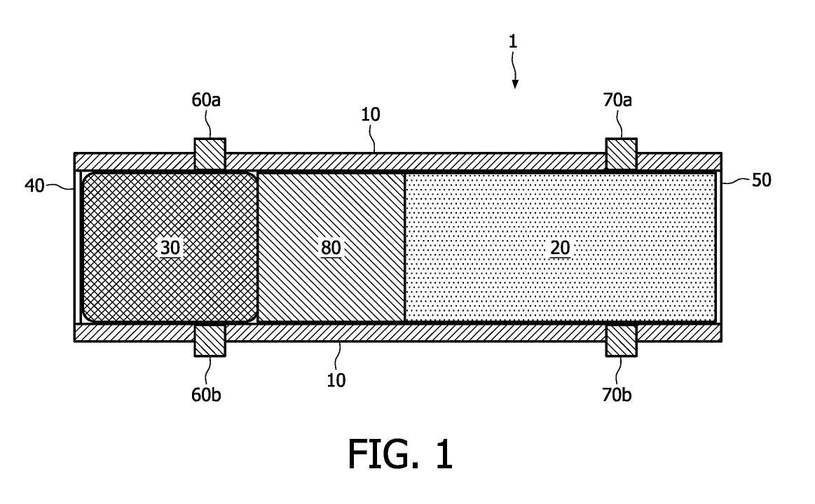

FIG. 1 shows a very schematical longitudinal cut-out view of an osmotic pump according to one embodiment of the present invention having a semipermeable membrane; and

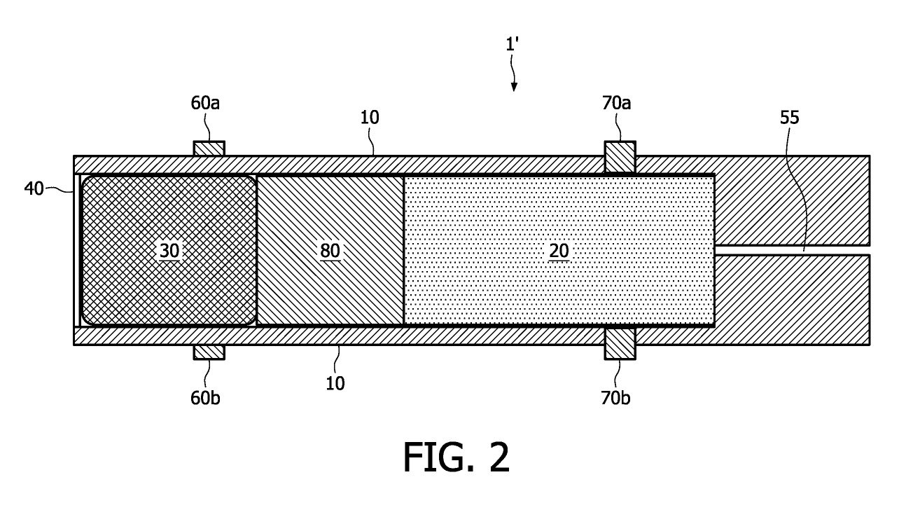

FIG. 2 shows a very schematical longitudinal cut-out view of an osmotic pump according to one embodiment of the present invention having a flow restrictor.

FIG. 1 shows a very schematical longitudinal cut-out view of an osmotic pump 1 according to one embodiment of the present invention. The pump 1 comprises a pump body 10, which may be in cross-cut section (not shown in the figs.) circular, elliptical, square- or rectangular. At both ends of the pump body there are semipermeable membranes 40 and 50.

The first semipermeable membrane 40 allows the inflow of water into a first reservoir, in which a solid chargeable material 30 is located (together with an water soluble chargeable material which is not shown). Upon applyment of current via the electrodes 60a and 60b, an osmotic pressure is induced and water will flow into the first reservoir. Thereby, the piston 80 will move towards the right and cause the drug solution 20 located in the second reservoir of the pump to be released via the second membrane 50.

In this embodiment, the pump is provided with two communication modules 70a and 70b which are able to communicate e.g. with an outside station. The communication modules 70a and/or 70b may also be equipped with pressure and temperature sensors and/or data collecting means which collect data concerning the amount of drug that has already been released via the osmotic pump 1.

The drug could be sufentanil, fentanil, morphine, leuprolide acetate, insulin, psychotropics, contraceptive agents, growth hormones or other proteins, peptides, enzymes, genes, factors and hormones.

By controlling the amount of current it is possible to raise or lower the osmotic pressure in the first reservoir. It should be noted that it is possible that—if the above-mentioned process is reversed, the chargeable material in the first reservoir is de-charged and therefore the osmotic pressure is reduced that far that water will leave the first reservoir via the membrane 40. This will cause the piston 80 to move to the left.

FIG. 2 shows a very schematical longitudinal cut-out view of an osmotic pump 1′ according to second embodiment of the present invention. The pump 1′ differs from the pump of FIG. 1 in that a flow restrictor (which is for visibility reasons very schematically drawn, in most actual applications the diameter of the flow restrictor will be quite smaller) 55 through which the drug is released.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and the equivalents thereto. Furthermore, reference signs used in the description and claims do not limit the scope of the invention as claimed.

1. An osmotic pump for drug delivery, especially for drug delivery to and/or inside a patient comprising at least one chargeable material wherein osmotic pressure is caused by inducing and/or de-inducing charges within the chargeable material.

2. The osmotic pump of claim 1 wherein said at least one chargeable material comprises a solid material.

3. The osmotic pump of claim 1 wherein said at least one chargeable material comprises water-soluble material.

4. The osmotic pump of Claim 1 herein the at least one chargeable material is located in a first reservoir comprising a first semi permeable material for the inflow of water into said reservoir

5. The osmotic pump of claim 1 wherein the osmotic pump comprises a second reservoir in which the drug to be released by the osmotic pump is located and wherein a movable piston is provided between the first reservoir and the second reservoir.

6. The osmotic pump of claim 1 wherein the second reservoir comprises a second semi permeable material through which the drug is released by the osmotic pump and/or a flow restrictor through which the drug is released by the osmotic pump

7. The osmotic pump of claim 1 wherein said at least one chargeable material in at least its charged form is selected from the group comprising alkylsulfonates, iodine, sulfides metals, Fe, Ni, Cu, Zn, Co, Al, Cr, Mo, Ru, Mn, Ir, Ag and mixtures thereof and their oxides, hydroxides, iodides, chlorides, sulfides, acetates, oxalates, phtalocyanines and mixtures thereof.

8. The osmotic pump of claim 1 wherein said at least one chargeable material is a solid material selected from the group comprising polypyrrole, polyaniline, polyacrylonitrile, polythiophene and mixtures thereof

9. A method of releasing a drug from an osmotic pump of claim 1 wherein osmotic pressure is induced by charging the at least one chargeable material by use of an electrical current.

10. A system comprising an osmotic pump according to claim 1, the system having one or more applications selected from the group consisting of:
drug delivery systems
liquid absorbers
sample handling devices
micro valves
analytical devices
micropumps
